SEQUENCE LISTING (8 sequences)

SEQ ID NO: 1 - DNA, 20 bp, Artificial Sequence
  Artificially designed polynucleotide
ccatgatatttcaatatatt

SEQ ID NO: 2 - DNA, 15 bp, Artificial Sequence
  Artificially designed polynucleotide
gtaagcgaactgata

SEQ ID NO: 3 - DNA, 15 bp, Artificial Sequence
  Artificially designed polynucleotide
gtaaccgacctgatc

SEQ ID NO: 4 - DNA, 15 bp, Artificial Sequence
  Artificially designed polynucleotide
ataagtgaactgaga

SEQ ID NO: 5 - DNA, 15 bp, Artificial Sequence
  Artificially designed polynucleotide
ggaagcgagcagata

SEQ ID NO: 6 - DNA, 20 bp, Artificial Sequence
  Artificially designed polynucleotide
cttgctttggcttgctgctt

SEQ ID NO: 7 - DNA, 20 bp, Artificial Sequence
  Artificially designed polynucleotide
ttggggattgactgggttcc

SEQ ID NO: 8 - DNA, 20 bp, Artificial Sequence
  Artificially designed polynucleotide
cagcccacccctctcacctg

(19) United States
(12) Patent Application Publication
(10) Pub. No.: US 2015/0310165 A1
(43) Pub. Date: Oct. 29, 2015

(54) EFFICIENT COMPARISON OF POLYNUCLEOTIDE SEQUENCES

(71) Applicant: ILLUMINA, INC., San Diego,, CA (US)

(72) Inventor: Tobias Mann, San Diego, CA (US)

(21) Appl. No.: 14/646,697

(22) PCT Filed: Mar. 7, 2013
(86) PCT No.: PCT/US2013/029653

§ 371 Date: May 21, 2015

Related U.S. Application Data
(60) Provisional application No. 61/729,791, filed on Nov. 26, 2012.

(51) Int. Cl. G06F 19/22; C12Q 1/68

(52) U.S. Cl. CPC G06F 19/22; C12Q 1/6869

(57) ABSTRACT

The disclosure relates to rapid detection of oligonucleotide sequence in a nucleic acid sequence database through the configuration of the database into rapidly searchable index classes built around perfect Hamming code oligonucleotides.

### FIELD OF THE INVENTION

The disclosure relates to rapid identification of oligonucleotides in nucleotide sequence datasets.

### BACKGROUND OF THE INVENTION

As high throughput nucleotide sequencing becomes a more routine tool in science and medicine, there is a need for rapid sequence analysis tools. In particular, there is a need for methods and devices that allow one to rapidly search for a large number of unique or fairly unique oligonucleotide sequences in a genomic data set. Current sequence analysis tools can take as long as 40 minutes or longer to identify a given set of relatively short polynucleotide sequences in a database of stored genomic sequences.

Some current approaches involve identifying markers within the genomic sequences in a database, and then creating an index of those markers. The system divides the sequence reads into short oligonucleotides, such as 15 mers, 20 mers or 25 mers for alignment against this index of stored genomic markers. However, for 25 million 20 mers that are taken from a sample sequence, it can take up to 40 minutes or more to align them to an indexed 65 million oligonucleotide marker set of stored genomic markers.

Alternately, one can generate an index of polynucleotide sequences that are being determined in a sequencing reaction and then search that index against a stored dataset of 65 million oligonucleotide markers from a genome. This process can take about 20 minutes of formatting and 20 minutes of searching at current computing capacity.

Neither of these options are particularly attractive, as both involve substantial amounts of time. Furthermore, current alignment tools such as “bowtie” or “bwa” are computationally intensive and require pre-built indices and extensive post-processing efforts. Additionally, current techniques relying on prebuilt techniques do not work well with customers that want to align their sequences against custom developed genomes. Building indices for custom genomes using present techniques slows the process even further and occupies substantial computer space.

### SUMMARY OF THE INVENTION

Some embodiments of the present disclosure comprise computer-based methods of searching for a first 20 mer oligonucleotide sequence in a nucleotide data set, wherein said sequence is homologous to and differs by at most one nucleotide from a second 20 mer oligonucleotide target sequence. In some aspects a method comprises selecting a second 20 mer oligonucleotide target sequence having a sequence which is unique in a reference genomic nucleotide data set and which spans one single nucleotide polymorphism. In some aspects a method comprises determining if the selected second 20 mer is present in a genomic sample to determine if the single nucleotide polymorphism is present in the sample. In some aspects a method comprises comparing said second 20 mer oligonucleotide sequence to a sequence determined from a sequencing reaction to determine whether the second 20 mer sequence is present in the sequences from the sequencing reaction. In some aspects a method comprises generating a report indicating whether said second 20 mer is present in said all or substantially all of said nucleotide sample sequence.

In some aspects a method further comprises initializing the sequencing reaction of a sample. In some aspects a method comprises comparing of the second 20 mer oligonucleotide sequence to the sequence determined from the sequencing reaction, and this comparing occurs prior to completion of the sequencing reaction.

In some aspects a method further comprises comprising making a decision to (a) perform further analyses on said all or substantially all of said nucleic acid sequence if said first 20 mer in said sequenced sample is identical to said second 20 mer or (b) halt further analyses on said all or substantially all of said nucleic acid sequence if said first 20 mer in said sequenced sample is not identical to said second 20 mer.

Some embodiments of the present disclosure comprise a method of aligning a 20 mer oligonucleotide sequence to all 20 mer fragments of a nucleic acid data set. In some aspects, embodiments comprise at least one of the following steps: 1) indexing said dataset into perfect Hamming code 5 mers, wherein each said 5 mer has a set of 15 5 mers which form an equivalence class such that each additional member of said class differs from said perfect Hamming code 5 mer by a Hamming code distance of 1, and wherein each said perfect Hamming code 5 mer differs from all other perfect Hamming code 5 mer by at least a Hamming code distance of 3; 2) configuring said indexed 5 mers into a trie having a branching factor of 64 such that each leaf of said trie represents the perfect Hamming code 5 mers of said dataset and the equivalence class it defines; 3) concatenating said trie such that each leaf of said index is the trunk of a second indexed 5 mer trie, each leaf of said second level trie is linked to a third level trie trunk, and each leaf of said third level trie is linked to a fourth level trie, such that said trie has a depth of 4 and 64⁴ leaves, and such that each 20 mer of said nucleic acids sample maps to only one path from a 4^{th} level leaf to the first level trunk of the trie; 4) assigning all 20 mer fragments of the nucleotide data set to a unique trie path corresponding to the 20 mer fragments; 5) identifying the trie path corresponding to the 20 mer oligonucleotide sequence; 6) comparing the 20 mer fragments of the trie path to the 20 mer oligonucleotide sequence; 7) comparing the 20 mer oligonucleotide sequence to the thirty equivalence classes at level 1 having at least on member which differs from at least one member of the equivalence class of the 20 mer oligonucleotide by a Hamming distance of 1; 8) comparing the 20 mer oligonucleotide sequence to the thirty equivalence classes at level 2 having at least on member which differs from at least one member of the equivalence class of the 20 mer oligonucleotide by a Hamming distance of 1; 9) comparing the 20 mer oligonucleotide sequence to the thirty equivalence classes at level 3 having at least on member which differs from at least one member of the equivalence class of the 20 mer oligonucleotide by a Hamming distance of 1; and 10) comparing the 20 mer oligonucleotide sequence to the thirty equivalence classes at level 4 having at least on member which differs from at least one member of the equivalence class of the 20 mer oligonucleotide by a Hamming distance of 1; such that all possible oligonucleotide sequences having a Hamming code distance of 1 from a 20 mer oligonucleotide sequence are analyzed through the selection of 121 out of a total of over 16 million equivalence classes in a four level index of concatenated 5 mers.

In some aspects the 20 mer oligonucleotide maps to a unique region of a genomic reference sequence and the oligonucleotide spans a single nucleotide polymorphic site.

In some aspects the method further comprises discarding the sample if the 20 mer oligonucleotide sequence is not identically present in the sample sequence. In some aspects the 20 mer oligonucleotide sequence is derived from a sample, and the nucleic acid data set is labeled to be derived from the sample.

In some aspects the nucleic acid data set comprises sequence from an incompletely sequenced nucleotide sample.

In some aspects the nucleotide sample is being sequenced concurrently with execution of the method.

In some aspects the method determines whether the nucleic acid data set and the 20 mer oligonucleotide sequence are derived from different sources.

In some aspects the 20 mer oligonucleotide sequence is determined by hybridization of a nucleic acid sample to a nucleic acid probe complementary to the 20 mer oligonucleotide at the single nucleotide polymorphic site.

In some aspects the 20 mer oligonucleotide sequence is determined using microarray hybridization. In some aspects the oligonucleotide sequence is determined using a sequencing method.

In some aspects a region comprising the oligonucleotide single nucleotide polymorphic site is amplified in a polymerase chain reaction.

In some aspects the 20 mer oligonucleotide maps to a region that undergoes copy number variation in a population, and hits to sequence identical to the 20 mer oligonucleotide are indicative of a copy number of the homologous region in the sample.

In some aspects the method further comprises searching the genomic sequence using a second 20 mer oligonucleotide sequence that is homologous to a region having a copy number that does not co-vary with the copy number variant region.

In some aspects the 20 mer oligonucleotide search results are indicative of the copy number of the homologous regions.

In some aspects a method comprises calculating a ratio of the copy number variation at a region.

In some aspects, alignment results may be used to determine the identity of duplicate nucleotide samples.

In some aspects multiple duplicate samples are mislabeled.

Some embodiments of the present disclosure comprise an oligonucleotide data set comprising oligonucleotide sequences. In some aspects the oligonucleotide sequences are unique in a reference nucleotide data set. In some aspects the oligonucleotide sequences span common single nucleotide polymorphic variants within the reference nucleotide data set, the oligonucleotides are separated from one another in sequence by a Hamming Code distance of at least 3, and the oligonucleotides have a length of five nucleotides, an integer multiple of five nucleotides, 21 nucleotides or an integer multiple of 21 nucleotides.

In some aspects the reference nucleotide data set is a substantially completed genome sequence.

In some aspects the reference nucleotide data set is a species reference genome.

In some aspects the species is human.

In some aspects the human reference nucleotide data set is Genome Reference Consortium human genome, for example build 37.

In some aspects oligonucleotides each have a length of 20 nucleotides.

Some embodiments comprise a method of verifying the identity of a polynucleic acid sample. In some aspects the method may involve one or more of the following steps: 1) providing a polynucleic acid sample; 2) contacting the polynucleic acid sample to a microarray, wherein the microarray comprises oligonucleotide probes that distinguish among single nucleotide polymorphisms of alleles within the sample; 3) obtaining an output from the microarray wherein the output is indicative of a distribution of single nucleotide polymorphisms in the sample; 4) entering the microarray output into a computer; 5) configuring the microarray output into an index of binned equivalence classes, wherein each class represents a 20 mer concatenation of four unique 5 mers, each of the 5 mer comprising a Hamming code sequence such that all single-nucleotide differences from the 5 mer map uniquely to only one 5 mer; 6) initiating a process of analyzing a polynucleic acid sample comprising contacting the nucleotide sample to a polynucleic acid sequencing device, wherein the polynucleic acid sequencing device produces an output comprising the sequence of the polynucleic acid sample, and wherein the process is performed over a duration of time; 7) receiving the output comprising the polynucleic acid sequence as the sequence is generated pursuant to the process; 8) entering the output comprising the polynucleic acid sequence into a computer as the sequence is generated pursuant to the process; 9) comparing the sequence to the binned equivalence classes, wherein the comparing comprises determining whether the sequence generated pursuant to the process is consistent with the output indicative of a distribution of single nucleotide polymorphisms in the polynucleic acid sample; and 10) ceasing the process comprising contacting the polynucleic acid sample to a polynucleic acid sequencing device if the determination indicates that the polynucleic acid sequence generated pursuant to the process is not consistent with the output indicative of a distribution of single nucleotide polymorphisms in the sample.

Some embodiments comprise a method of verifying the identity of a nucleotide sample. In some aspects the method may comprise one or more of the following steps: 1) providing a polynucleic acid sample; 2) determining an oligonucleotide array output of the sample; 3) inputting the output into a computer; 4) configuring the output as an index of binned equivalence classes; 5) generating a sequence of at least some of the polynucleic acid sample; 6) comparing the sequence with the binned equivalence classes; 7) determining whether the polynucleic acid sequence is consistent with the output configured into the index of equivalence classes; and 8) performing a further analysis upon the nucleotide sample if the sequence is consistent the output configured into the index of equivalence classes.

In some aspects the index of binned equivalence classes comprises 20 mer oligonucleotide sequences, wherein the 20 mers comprise concatenated 5 mer oligonucleotide sequences, and wherein the 5 mers comprise perfect Hamming code sequences such that each 5 mer in the generated sequence either coincides with only one of the perfect Hamming code sequences or differs from only one the unique perfect Hamming code sequences by a single base

In some aspects the index comprises about 16 million equivalence classes

In some aspects the comparing to the index of binned equivalence classes comprises a number of steps that is proportional to O (N) and wherein the number of steps is not directly proportional to O (N log N).

In some aspects the comparing to the index of binned equivalence classes comprises examining a subset of the 16 million equivalence classes.

In some aspects the subset consists of 121 of the 16 million equivalence classes.

Some embodiments of the present invention comprise an index of oligonucleotide sequence data, wherein the index comprises about 16 million equivalence classes, each equivalence class comprising a concatenated set of four 5-mer oligonucleotide sequences, each 5-mer oligonucleotide sequence being selected such that all possible 5-mer oligonucleotide sequences are either identical to or differ by exactly one base from the selected 5-mer oligonucleotide sequences.

Some embodiments of the present invention comprise A computer-based system for evaluating an input polynucleic acid sequence comprising a dataset indicative of the polynucleic acid sequence, wherein the dataset is configured as the index of claim 8; an input polynucleic acid sequence, wherein the input polynucleic acid sequence comprises at least part of a complete sequence of a polynucleic acid sample; a selection module capable of selecting a subset of 121 equivalence classes of the index for comparison to the input polynucleic acid sequence; a comparison module capable of comparing the input polynucleic acid sequence to the oligonucleotides of the index; and a report module capable of reporting the results of the evaluation to a user.

### BRIEF DESCRIPTION OF THE DRAWINGS

FIG. 1 is a block diagram of an embodiment of a polynucleotide comparison system.

FIG. 2 is a flow diagram of one embodiment of a process for comparing nucleotide sequences.

FIG. 3 is a block diagram of an equivalence class according to one embodiment. The sequence GCGTT at the center is a Perfect Hamming Code (PHC) of the illustrated equivalence class.

FIG. 4 is a diagram of a four level trie of indexed PHC sequences made of five nucleotides (5 mers). Only 3 of the 64 leaves at each level are shown, and only a single leaf at each level is shown to form the trunk of the next level. The figure depicts the path representing the 20 mer CCATGATATTTCAATATATT (SEQ ID NO:1). The actual trie would have over 16 million leaves.

FIG. 5 is a graph showing the results of a copy number determination through an embodiment of the method.

FIG. 6 is a block diagram showing a simplified depiction of three 20 mer leaves of a one-level trie (the other 61 leaves are not shown). The sequences listed are as follows: CTTGCTTTGGCTTGCTGCTT (SEQ ID NO:6); TTGGGGATTGACTGGGTTCC (SEQ ID NO:7); CAGCCCACCCCTCTCACCTG (SEQ ID NO:8). Also shown in FIG. 6 is an all by all comparison of a needle index with a haystack index.

FIG. 7 is a simplified block diagram depicting two equivalence classes having a Hamming code distance of 3 to one another.

FIG. 8 shows one embodiment of a process for filling input buffers and processing results in a sequence comparison system.

FIG. 9 is a block diagram showing an embodiment of a process for determining results of a search for polynucleotide “needles” within a target “haystack” database of sequences.

### DETAILED DESCRIPTION

The present disclosure relates to methods, systems and datasets that are configured to rapidly search for oligonucleotide sequences in a dataset of polynucleotides, such as a genomic sequence dataset. Some embodiments include a system for rapidly determining if a DNA sample being sequenced is the correct sample. In this embodiment, a biological sample of DNA is provided. That DNA is processed according to known methods and run over a microarray to identify unique single nucleotide polymorphisms (SNPs) within the biological sample. That data, from 100, 1000, 10,000, 100,000, 1,000,000, or more SNP sites in the biological sample is stored in the analysis system, or laboratory information management system (LIMS). Thus, SNP data for that particular biological sample is now known. In this embodiment, the same biological sample is then run on a sequencer to determine the exact DNA sequence of some or all of that user's DNA. However, during the sequencing reaction, it is possible that incorrect samples are applied to the sequencer, or other errors are made so that the sequencer is not properly sequencing the desired sample. Thus, this system provides a way of checking, in real time, whether the proper sample was placed on the sequencer.

As the sequence data is being generated by the sequencer, there are hundreds, thousands, tens of thousands, or more sequencing reactions occurring at the same time in the sequencer. In embodiments of the invention, each of these reactions is monitored, and oligonucleotide sequences from the sequencer are compared to the sequences found in the earlier microarray data to ensure that there is concordance. Thus, the same person would have the same unique SNPs found in their DNA from the microarray experiment as would be found in the sequence data. Should the system find that a particular unique SNP generated from the sequencer did not match with the same SNP determined from the microarray, the system could set an error flag and notify the operator that a sample mistake may have been made. This can prevent the wrong sample from being sequenced, and save on valuable reagents that would otherwise have been used to sequence the incorrect sample.

A challenge addressed by embodiments of the present disclosure is that of comparing millions of unique sequences against a panel of millions of unique SNP regions in a fast, and even real time manner in order to detect errors. In some embodiments, the described system can align a dataset of up to 65 million unique polynucleotides against a set of 25 million 20 mer oligonucleotides in less than 20 minutes. In one embodiment, the polynucleotides in the dataset and the oligonucleotides are both 20 nucleotides in length (“20 mers”). Smaller-scale applications, such as matching 20-30 20 mer oligonucleotide probes against a genomic sequence dataset of 65 million polynucleotides can be accomplished in less than 1 minute.

In some embodiments, disclosed herein are methods, processes and computer-based devices for the rapid identification of oligonucleotide ‘needles’ in large polynucleotide datasets, termed herein ‘haystacks’. In some embodiments this rapid identification is effected through a process of rapid, efficient indexing followed by a greatly facilitated comparison of the needle sequences to the indexed haystack sequences.

Perfect Hamming Code 5 mers

In some embodiments the system uses the mathematical concept of perfect Hamming codes to index a set of oligonucleotides into an index that has the properties of a perfect Hamming code (‘PHC’). In this embodiment, for example, a set of 64 oligonucleotide sequences that are five nucleotides in length (5 mers) can be generated wherein every 5 mer oligonucleotide sequence not in the 64 5 mer set differs from one and only one of the other 64 5 mers in the perfect Hamming code set by one and only one base substitution, and has more base substitutions when compared to all other members of the 5-mer set. See Takenaka, et al, (2011) “Perfect Hamming code with a hash table for faster genomic mapping” BMC Genomics 12(53):58. This property of forming a perfect Hamming code is unique to oligonucleotides of length k, wherein (3^{k}+1)/4 is a whole number. Nontrivial solutions for this equation include 5 and 21.

The oligonucleotides which differ from one of the 64 Hamming code solution 5 mers by a single base change (that is, having a ‘Hamming distance’ of 1) may be described as falling into the same equivalence class as that of the Hamming code solution 5 mer. See Example 1, depicting equivalence classes for three perfect hamming code (PHC) 5 mer sequences, which also include the PHC 5 mer itself.

A result of this structure yields an approach to search for approximate matches to 5 mer sequences on the basis of the equivalence classes generated by the perfect hamming code. If the system must find 5-mers with at most one mismatch to a query 5 mer, it can do this on the basis of the equivalence classes by examining equivalence classes whose perfect hamming code template has a hamming distance of 3 to the perfect hamming code template for the original query 5 mer. One property of the perfect hamming code is that each member will have hamming distance 3 to exactly 30 of the 64 members of the perfect hamming code. Restated, a non PHC 5 mer in a given equivalence class (having a Hamming distance separated from the PHC 5 mer of its equivalence class by 1) can only match sequences with at most one error if those sequences are in one of 30 equivalence classes defined by the PHC template for the original query sequence.

### Constructing Indices of Concatenated 5Mers

In some embodiments, however, it may be advantageous to align sequences that are longer than five nucleotides when searching for unique genomic sequences. Thus, embodiments of the system have been developed that concatenate 5 mer PHC oligonucleotides into longer sequences of 10 mers, 15 mers, 20 mers, 25 mers or larger sequences in intervals of 5 nucleotides. In some embodiments, a concatenated 20 mer oligonucleotide made up of four sets of Hamming code solution 5 mers, for which each individual 5 mer falls into an equivalence class of the corresponding Hamming code 5 mer used to form the concatenated 20 mer, may be described as falling into the same equivalence class as the concatenated 20 mer of four Hamming code solution 5 mers. In this embodiment, a 20 mer made from four PHC 5 mers may differ by as many as four bases from the members of its equivalence class, so long as there is only one difference in the first 5 base interval, 1 in the second 5 base interval, one in the third 5 base interval, and 1 in the fourth 5 base interval, each interval corresponding to a PHC 5 mer from which the concatenated 20 mer was built.

In some embodiments the 64 sets of equivalence classes each corresponding to a PHC 5 mer can be depicted as endpoints, or ‘leaves’, branching from a single trunk of a decision ‘trie’. Thus any 5 mer at the base of the trie can be placed into the bin represented by a leaf at the end of each branch based on having a sequence within the equivalence class of that leaf.

In some embodiments each endpoint leaf of this first branch level in turn serves as a trunk for a new set of 64 branches. Thus the second level of the Vie′ has 64×64, or 4096 leaves as endpoints. Thus any 10 mer can rapidly be mapped to a single level two leaf, based on the placement of its first 5 mer into an equivalence class of a leaf at the first level, and then the placement of its second 5 mer into a leaf that branches from the leaf/trunk of the first level equivalence class.

Notably, although there are 4096 equivalence class leaves for this two-level tree representing all possible 10 mers, only 128 leaves need to be evaluated—64 form the first level and then the sixty four of the second level that branch from the equivalence class leaf identified from the first level. Thus the configuration of the sequence space of all possible 10 mer sequences into equivalence classes based on concatenated PHC 5 mer leaves dramatically reduces the amount of sequence space that one needs to search to place a given 10 mer into an equivalence class consisting of 10 mers which differ from it by as many as 2 bases.

In some embodiments each endpoint leaf of the second branch level in turn serves as a trunk for a new set of 64 branches. Thus the third level of the ‘trie’ has 64×64×64, or 262144 leaves as endpoints.

Any 15 mer can rapidly be mapped to a single level three leaf, based on the placement of its first 5 mer into an equivalence class of a leaf at the first level, placement of its second 5 mer into a leaf that branches from the leaf/trunk of the first level equivalence class, and placement of its third 5 mer into a leaf that branches from the leaf/trunk of the second level equivalence class.

Similarly, in some embodiments each endpoint leaf of the third branch level in turn serves as a trunk for a new set of 64 branches. Thus the fourth level of the ‘trie’ has 64×64×64×64, or 16,777,216 leaves as endpoints.

However, any 20 mer can rapidly be mapped to a single level four leaf, based on the placement of its first 5 mer into an equivalence class of a leaf at the first level, placement of its second 5 mer into a leaf that branches from the leaf/trunk of the first level equivalence class, placement of its third 5 mer into a leaf that branches from the leaf/trunk of the second level equivalence class, and placement of its fourth 5 mer into a leaf that branches from the leaf/trunk of the third level equivalence class.

Thus, placement of a 20 mer into one of over 16 million equivalence classes may be accomplished by evaluating a mere 256 leaf/5 mer equivalence classes—64 at leach level.

### Comparing Leaves of Multiple Trie Indices

In some embodiments of the present disclosure, nucleotide data sets may be divided into concatenated 5 mers and configured into multilevel trie indices. In a preferred embodiment the concatenated oligonucleotide length is a 20 mer.

In some embodiments a configured nucleotide data set ‘haystack’ is to be compared to a set of oligonucleotide probe or ‘needle’ sequences. In some embodiments both the haystack and the needle sequence data sets are configured into ‘trie’s of equivalence classes, such that the fourth level ‘leaves’ may be rapidly compared to one another.

In some embodiments 20 mers differing from a needle 20 mer by at most 1 single nucleotide may be detected, such as 20 mers which span a single SNP which may be indicative of the identity of a nucleotide sample. This comparison involves, for a single 20 mer needle sequence, analysis of a number of hay leaf/equivalence classes equal to the 1) equivalence class of the 20 mer needle itself, 2) the 30 equivalence classes having a 5 mer in their first position that differs by one from a non PHC 5 mer of the needle first position 5 mer's equivalence class, 3) the 30 equivalence classes having a 5 mer in their second position that differs by one from a non PHC 5 mer of the needle second position 5 mer's equivalence class, 4) the 30 equivalence classes having a 5 mer in their third position that differs by one from a non PHC 5 mer of the needle third position 5 mer's equivalence class, and 5) the 30 equivalence classes having a 5 mer in their fourth position that differs by one from a non PHC 5 mer of the needle fourth position 5 mer's equivalence class.

Thus, for example, an indexed 20 mer needle may be matched to a sequence in a leaf of an indexed haystack trie of 20 mer sequences by examining a mere 121 sequences out of the over 16 million haystack trie leaf bins.

Identifying Useful Needle Sets

In some embodiments as disclosed herein, a set of 65 million 20 mers is evaluated against a reference data set such as the human genome to identify 20 mers that are unique or fairly unique to a genomic data set and that span variable regions such as single nucleotide polymorphisms. Such 20 mer oligos have as a characteristic the trait that they differ from a homologous sequence in another individual's genome by a Hamming distance of 1.

This reduced set of 20 mers has the novel, useful trait that their homologous sequence in an unknown sample can be identified in an indexed human genome dataset by evaluating the contents of only 121 leaf/equivalence class bins out of the over 16 million theoretically extant bins in such a dataset.

### Implementations of the Methods, Indices and Computer-Based Search Algorithms Disclosed Herein

Given this refined set of 20 mer oligonucleotide sequences, one can rapidly assay nucleotide sequence data, such as genomic sequence as it is being generated in a sequencing run, to determine whether or not the sequence data is properly labeled and identified. Such a technique is a valuable tool for the identification of, for example, mislabeled genomic samples early in the sequencing process to prevent the loss of further reagents, computing capacity and time. The technique can be valuable in other contexts as well including, but not limited to, forensics (e.g. identification of individuals based on nucleic acid sequence similarities to one or more forensic reference sequence), diagnostics (e.g. determining risk of a clinical condition or disease based on nucleic acid sequence similarities to one or more diagnostic reference sequences) or prognostics (e.g. predicting a clinical outcome based on nucleic acid sequence similarities to one or more prognostic reference sequences), or metagenomics (e.g. identifying the species composition of microbial mixtures), or DNA barcoding (e.g. identifying organisms based on unique tag sequences)

In some embodiments, sequence comprising a useful set of 20 mer selected so as to map to a unique region of the human reference genome and to span a single SNP are selected. In some embodiments a microarray comprising at least one of these 20 mer sequences, or a subset thereof sufficient to distinguish the identity a nucleotide at said SNP in a sample, is contacted to a sample under conditions known in the art to facilitate determination of the SNP nucleotide at said position in said 20 mer sequence of said sample. In some embodiments the identity of a single SNP in a genomic sequence spanning a 20 mer of interest is determined using methods other than microarray analysis, such as sequencing of a polymerase chain reaction generated amplicons spanning the SNP, or differential hybridization of a probe to a polymerase chain reaction generated amplicons spanning the SNP. Other methods of determining the identity of a nucleotide at an SNP position are consistent with the methods disclosed herein.

In some embodiments a 20 mer corresponding to the sequence of the useful 20 mer with the SNP position specified is used as a needle to assay for the presence of said sequence, or to identify single nucleotide variants of said sequence, in a genomic sample. In some embodiments, the sequence to be assayed is believed to be derived from the same sample that was applied to a microarray, such that the identification of the specified 20 mer sequence may be used as a means of confirming that the sample being sequenced is correctly labeled (or otherwise correctly correlated or cross-referenced with the sample used for microarray analysis).

In some embodiments, the 20 mer searching process is fast enough so that a verification of the identity of a sequence may be made while the sequencing of the sample is in progress. In some embodiments a sequencing reaction such as a whole-genome sequencing reaction being performed on a sample may be terminated if said sample is determined by the methods disclosed herein not to match its expected sequence. In some embodiments, genomic sequence determined by the methods disclosed herein not to match its expected sequence is withdrawn from further analysis, for example under suspicion that said genomic sample has been mislabeled. Alternatively, the sequencing reaction can be paused, for example, while sample tracking is sorted out, and subsequently restarted. For diagnostic, prognostic or forensic applications, sequencing can be terminated or paused once similarity between test and reference nucleic acid sequences has been determined at a desired or predetermined confidence level.

In some embodiments the method disclosed herein may be used to evaluate copy number variation in a genomic sample. Copy number variation is presently determined using, for example, differential hybridization intensities on microarrays. In some embodiments of the present invention, 20 mer oligonucleotide sequences homologous to genomic regions where relative copy number determinations are desired may be selected. Genomic sequence for which copy number determinations are to be made may be indexed and searched using the 20 mer needles as discussed. In some embodiments, the number of hits to a given 20 mer needle in a genomic sample sequence is indicative of the relative copy number of that genomic region. Accordingly, methods set forth herein can be used to identify an aneuploidy, for example, in a fetal nucleic acid sample obtained from a pregnant female, or in a nucleic acid sample obtained from an individual suspected of having cancer.

Using the methods of the present disclosure, copy number is evaluated using quantitative digital methods and discrete statistics, rather than quantification of analog signal processing data.

In some embodiments the copy number determined corresponds to the ploidy of a given genomic region or locus. In some embodiments the region is of substantial significance to the health of an individual with which a sample is associated. For example, aneuploidy detected for a fetal nucleic acid sample can be indicative of mental retardation or other developmental delays. Aneuploidy can also be indicative of cancer.

In some embodiments, the methods disclosed herein may be used to correctly identify a number of samples which are being run simultaneously and for which the identities of the samples have been confused, for example by adding a sample to the wrong flow cell in a multiplex sequencer. In some embodiments the samples are run in duplicate. In some embodiments 20 mer oligonucleotides may be used to identify homozygous mismatches with the human genome reference sequence in a given sample that are distinctive to a given pair of samples, such as homozygous disagreements to a reference, such that all samples can be accurately paired.

### Operational Parameters of the Methods and Computer-Based Search Algorithms Disclosed Herein

In some embodiments, the computational methods disclosed herein may be performed on a graphics processing unit. In some embodiments the computational methods disclosed herein operate from bcl files. In some embodiments operation of the computer-based methods disclosed herein operate on bcl files and require no intermediate file generation. In some embodiments there may be separate implementations for full and sparse indices, or for GPU and multi-core searchers. In some embodiments uncompressed, gzipped or bzipped FASTA and FASTQ files may be read. In some embodiments, algorithms are designed to parallelize well for short reads or long chromosomal sequences. Some embodiments comprise seqanized design style and/or highly parallelized IO/searching.

In some embodiments, the computational burden of performing the methods and computer based algorithms disclosed herein scales as O (N) rather than O (N log N) in methods available in the art. Consequently, the methods and computer based algorithms disclosed herein may be performed more rapidly with substantially less computer computational capacity and memory than methods and computer based algorithms known in the art. This advantage is particularly true for large values of N, wherein N is the number of 20-mers to be analyzed—the sorting efficiency is of greatest benefit when there are a lot of needles or a lot of haystacks to analyze.

### Operational Low Level Architecture of the Methods Disclosed Herein

In some embodiments, sequences are split into 20 mer oligonucleotide needle sequences and then binary encoded, for example such that the bases a, c, g, and t correspond to 00, 01, 10, and 11, respectively. 20 mers may be encoded as 64 bit unsigned integers, and each short unsigned integer may contain a 5 mer, such that each encoded unsigned short integer is also a number from 0 to 1024.

For an arbitrary 20 mer, a node index may be computed as follows. Sort the 64 PHC template 5 mers and the associated 5 mers of each equivalence class. Place the first 5 mer of the arbitrary 20 mer into an equivalence class by evaluating the 1024 5 mers in that set. Then repeat the process for the second 5 mer, third 3 mer and fourth 5 mer, placing each into a 5 mer equivalence class so that the arbitrary 20 mer can be placed into four concatenated equivalence classes.

To perform a Hamming distance 1 search on a given node index (that is, to identify all oligonucleotides which differ from a given oligonucleotide by 1), one may proceed as follows. Reconstruct the concatenated 5 mers constituents of said node index. Then compute a lookup table for each 5 mer wherein said table comprises all of the 30 Hamming distance 1 search neighbors of said 5 mer sequence. The data may be processed by replacing bits and computing the resulting leaf offset, and adding the computed offset to a list. The process may be repeated for each 5 mer in a concatenated oligonucleotide such as a 20 mer.

Building this neighbor list requires a small number of integer additions and table lookups. For a 20 mer, a neighborhood of 121 equivalence classes must be evaluated to determine the set of Hamming distance 1 oligonucleotides. Keeping in mind that there are over 16 million 20 mer equivalence classes, reduction of the number to be searched to 121 represents a major improvement over current methods.

A workflow arising out of the methods and computational algorithms disclosed herein is as follows. For a given needle index and haystack index generated using the methods and computer based algorithms disclosed herein, a workflow comprises filling an input buffer and initializing a results buffer, processing the buffers on CPUs, and reading and processing the results buffer. Alternately, one may copy buffers to a GPU, process the buffers on a GPU, copy the buffers from a GPU, and read and process the results buffer.

The search buffers for a neighborhood search may be set up by using a needle index to copy the needle 20 mers into a local buffer and using a haystack index to copy the haystack 20 mers into a local buffer. A local results buffer may be initialized wherein each needle index is compared to all calculated haystack indices. The sequences of each indexed 20 mer needle/haystack pair may be compared directly or their integer values calculated above may be compared using the ‘xor’ operation. A difference of 0 indicates that the sequences are identical. A difference of 1 in integer value indicates a Hamming distance of 1, thus 1 base difference between the oligos. A difference of 2 may indicate a Hamming distance of 1 or 2, and may be further investigated to determine the actual Hamming distance. A difference of 3 or greater indicates a Hamming code distance of greater than or equal to 2.

In cases where the difference is 2, a Hamming distance of 1 may be found if the nucleotide substitution involves substitutions with two-bit changes as discussed above. The possible changes are A/T (00 to 11) and G/C (10 to 01). These situations can be distinguished from other situations resulting in a difference of 2 by ensuring that the least significant nonzero bit in the xor result is at an even position, and the adjacent higher bit is set.

For example, if analysis indicates that the lowest nonzero bit is odd, then the difference cannot result from a single substitution. Similarly, if the next bit is not set, then we cannot be looking at a single substitution.

Further understanding of embodiments of the disclosure may be had by a more detailed examination of the accompanying figures.

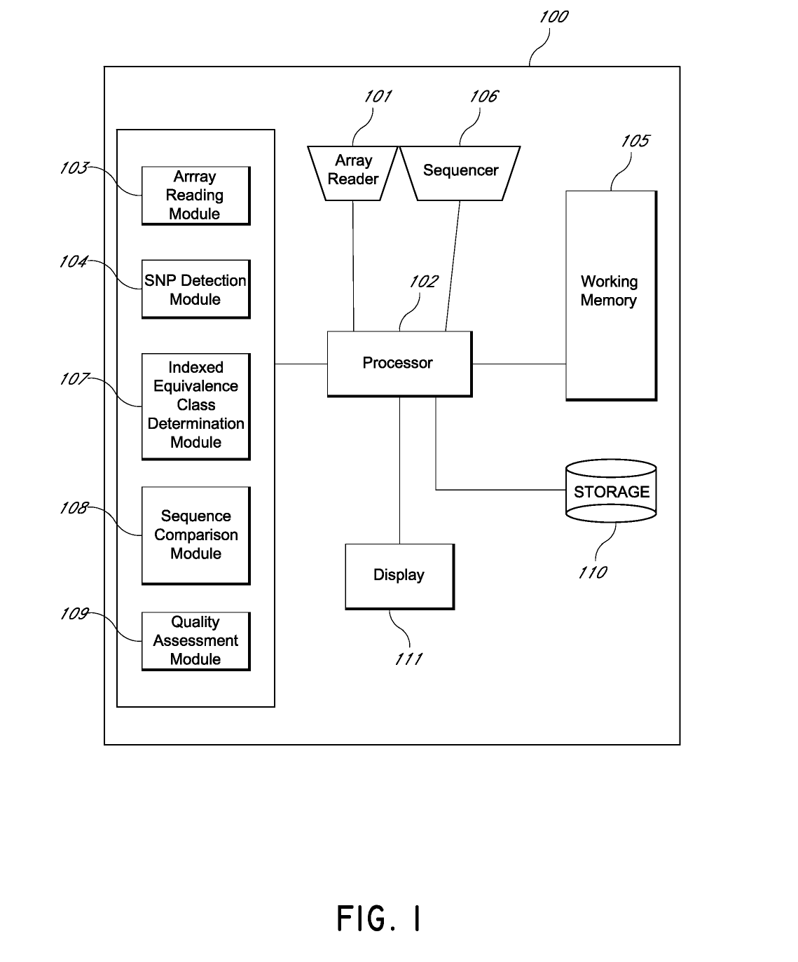

FIG. 1 depicts a system 100 configured to carry out some embodiments of the present disclosure. The system 100 includes a connection to an array reader 101 that can read microarray hybridization results. Microarrays and array readers known in the art may be used, for example the Illumina iScan, HiScan® or BEADEXPRESS® system. The microarrays to be read may comprise oligonucleotide sequences that selectively bind to specific alleles at single nucleotide polymorphic (SNP) sites. Thus in some embodiments the array reader will receive signals indicative of the identity of a base at an SNP position in a given oligonucleotide being assayed, such as a 20 mer spanning an SNP in an otherwise unique region of a reference genome. In one embodiment, the sequence is a human genome reference sequence, meaning that the sequence is unique in the genome of human beings and includes at a polymorphic region that differs by a single base pair between different alleles. In some embodiments the oligonucleotide sequence on the microarray is configured to selectively bind to a particular SNP base but is not the same length as the selected 20 mer to be used in downstream processes, as discussed below.

Data from the Array Reader 101 is passed to a Processor 102 which evaluates and processes any data arriving from the array. Stored within a memory of the system 100, and configured to be ready the processor 102, is an Array Reading Module 103 which converts the signal intensities reported by the Array Reader 101 into digitally accessible data. This data may be presented to a SNP Detection Module 104 which identifies the base at the SNP position or positions of interest. The SNP position data, as well as 20 mer full length data, may be stored on a Working Memory module 105 connected to the Processor 102.

A Sequencer 106 may also be in communication with the Processor 102. The sequencer may generate nucleic acid sequence data, such as genomic sequence data, from a biological sample of DNA taken from a human being. In some embodiments the sample from which Array data is taken is the same sample that is being sequenced, such that the data being sequenced can be confirmed by the data gathered from the microarray. The data generated by the sequencer may be provided to the processor for immediate analysis and may be concurrently or later stored on the Working Memory 105.

An Indexed Equivalence Class Determination Module 107 may also be present in the system 100, and configured to run on the Processor 102. The Indexed Equivalence Class Determination Module 107 may include instructions for identifying 64 member PHC 5 mer sets of oligonucleotides. The module 107 can also identify, for each PHC member, the remaining 15 5 mers that have a perfect Hamming code distance of 1 from each PHC 5 mer. The Indexed Equivalence Class Determination Module may also serve to concatenate PHC 5 mers to generate longer oligonucleotides. In some embodiments, the Indexed Equivalence Class Determination Module is configured to concatenate four of the PHC 5 mers together to generate a 20 mers oligonucleotide that is used for further analysis. The Indexed Equivalence Class oligonucleotides may be analyzed directly or may be stored in the Working Memory 105.

A Sequence Comparison Module 108 may also be included within the system 100, and configured to run on the Processor 102. The Sequence Comparison Module 108 may perform a sequence comparison, either directly or through the analysis of digital depictions of the oligonucleotide data, such as the sequence comparison approaches disclosed herein.

A Quality Assessment Module 109 may also be included within the system 100 and configured to run on the Processor 102. The Quality Assessment Module 109 may include instructions to evaluate the results generated by the Sequence Comparison Module 108 to generate a report indicating whether a particular 20 mer oligonucleotide sequence determined by contacting a sample to an Array Reader 101 is consistent with the results obtained by contacting a sample to a Sequencer 106, as mentioned above. The Quality Assessment Module 109 may be configured to read the SNP results determined from a sample read of the microarray, and compare those results with a sample being sequenced on the sequencer 106. This allows a real time analysis of whether the sequencer is properly sequencing a predetermined sample. For example, if the microarray data indicates that a particular person has a SNP within a unique position in his or her genome, that data can be compared to sequence data coming from the sequencer 106. If that unique position is also being sequenced, then a comparison can be made to determine if the SNP in the sequenced sample matches with the SNP found in the same person's DNA when it was placed on the microarray. If the samples are accurate, then every unique SNP position from the microarray will match when that same unique section is sequenced on the sequencer 106. However, if a determination is made that the SNP sequences don't match, then the process can be flagged, paused, terminated or halted since the samples do not match one another, when they should.

The system 100 also includes a storage 110 which is configured to electronically store sequence information, Indexed equivalence classes, PHC 5 mers, Array data and any other date involved in this process may be stored in the Storage 110. The system 100 also includes a display 111 for displaying results to a user.

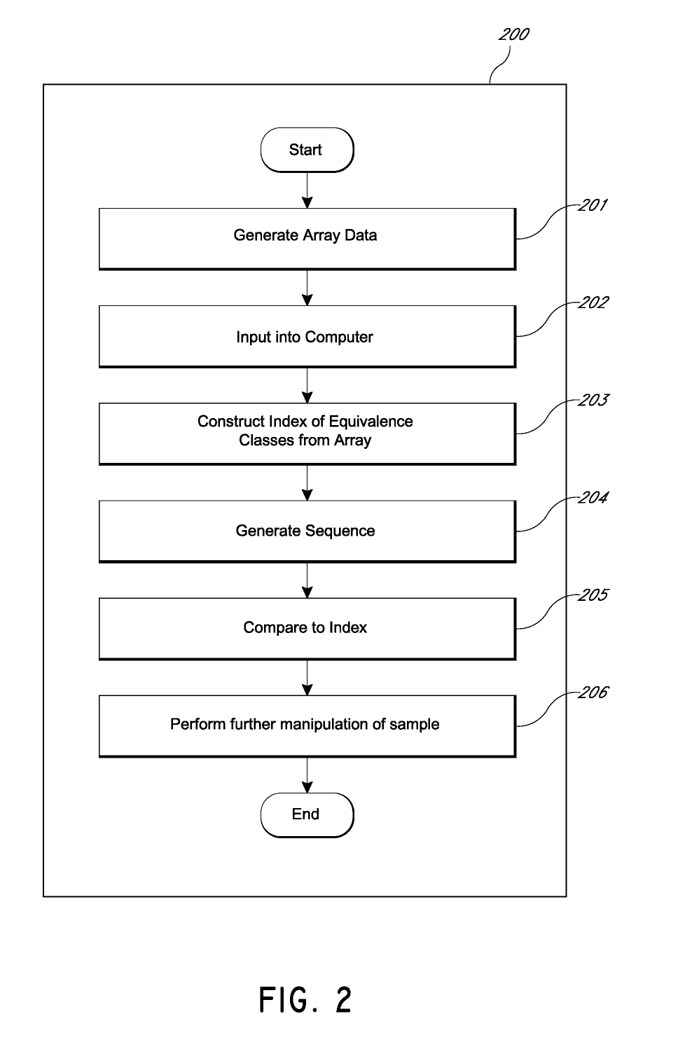

FIG. 2 depicts a flow diagram of a process 200 of how to compare indexes of two samples through steps of a method consistent with some embodiments disclosed herein. At a state 201, array data is generated, which are indicative of the identity of one or more nucleotides in, for example, a 20 mer from a particular biological sample which maps to a unique region of a genome and spans a single SNP, in a nucleic acid sample of interest. This array data is then inputted or read by a computer at a state 202. After the data is read by the computer, an index of equivalence classes is created at a state 203, informed by the sequence information from the array. The same biological sample can then be run on a sequencer to generate sequence data at a state 204. After, or at the same time that the sequencer is determining DNA sequences from the biological sample of interest, the process 200 moves to a state 205 wherein the sequence is compared to the Index generated at state 203 to determine whether the data from the array is consistent with the data from the sequencer. Depending on the results of this comparison carried out at state 205, one may then perform further manipulation to the sample at a state 206, such as generating more sequence data, performing post-sequencing analysis of the sequence, or discarding the sample and the sequence if the sequenced DNA does not seem to be matching the expected DNA sequences.

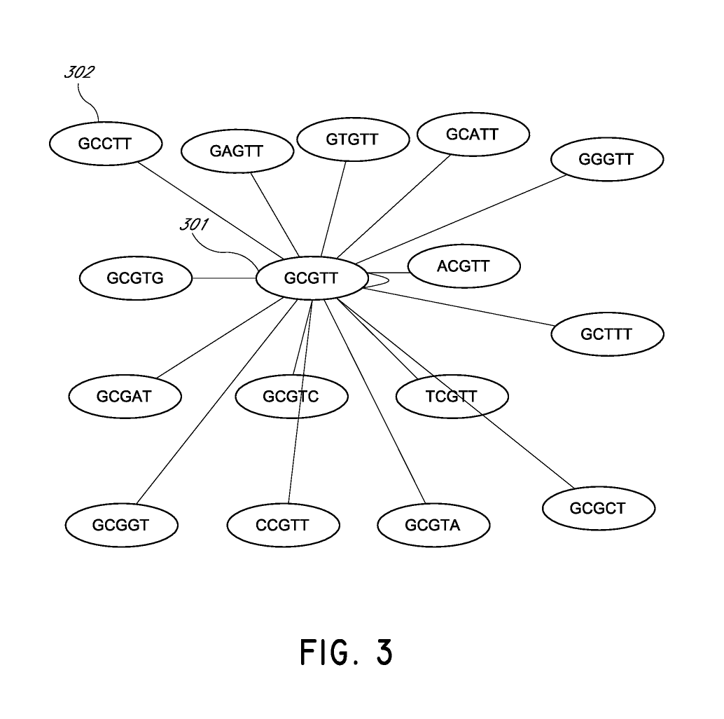

FIG. 3 depicts an Equivalence Class built around a PHC 5 mer 301, having a nucleotide sequence GCGTTT. All members of the class, aside from oligo 301, differ from oligo 301 by no more than a single base. That is, each has a Hamming distance of 1 from the PHC oligo 301. Oligo 302, having a nucleotide sequence of GCCTT, for example differs from the PHC oligo 301 by a single G/C change at position 3 of the 5 mer. PHC 5 mer 301 differs by all other PHC 5 mers by at least a Hamming code distance of 3, while other members of the equivalence class may differ from members of other equivalence classes by a Hamming code distance of 1.

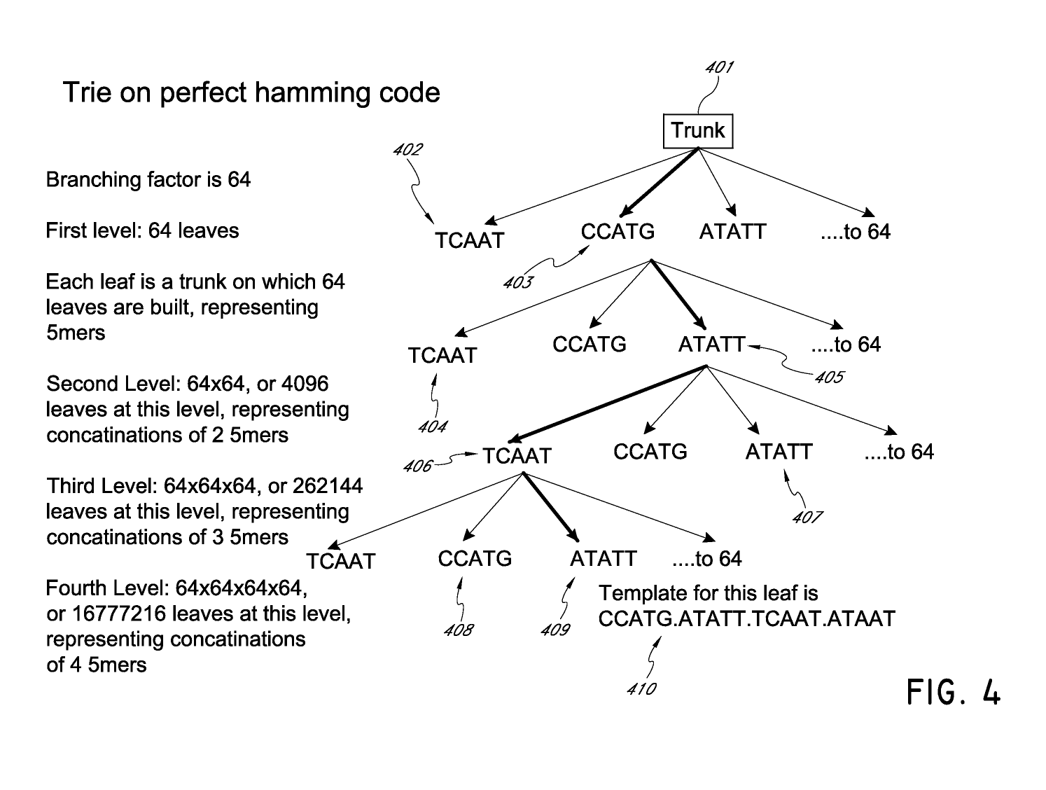

FIG. 4 is a partial depiction of a four-level PHC trie used to rapidly analyze 20 mer polynucleotide sequences. As is known to those of ordinary skill in the art, a “trie” is an ordered tree data structure that is used to store a dynamic set, or associative array, of data strings, such as nucleotides. Unlike a binary search tree, no node in the trie stores the key associated with that node. Instead, the data position in the tree defines the key with which it is associated. All the descendants of a node have a common prefix of the string associated with that node, and the root is associated with the empty string. Values are normally not associated with every node, only with leaves and some inner nodes that correspond to keys of interest. The trie starts from a Trunk 401, from which all 64 PHC 5 mers directly branch to form the first-level leaves (402, 403, for example) of the trie. Only three of the 64 PHC leaves at this level are depicted.

In this depiction, first level leaf node 403 is shown as the trunk of a second level branching of 64 PHC 5 mers (404 and 405 are examples). Only three of the 64 PHC leaves at this level branching from 403 are depicted, and none of the 63 other branching sets built off of the 63 other first level leaves are depicted.

Second level leaf 405 is shown as the trunk of a third level branching of 64 PHC 5 mers (406 and 407 are examples). Only three of the 64 PHC leaves at this level branching from 405 are depicted, and none of the other branching sets built off of the (64²−1) other second level leaves are depicted.

Third level leaf 406 is shown as the trunk of a fourth level branching of 64 PHC 5 mers (408 and 409 are examples). Only three of the 64 PHC leaves at this level branching from 406 are depicted, and none of the other branching sets built off of the (64³−1) other second level leaves are depicted.

Also depicted is the path through 403, 405, 406 and 409, representing 20 mer oligonucleotide 410.

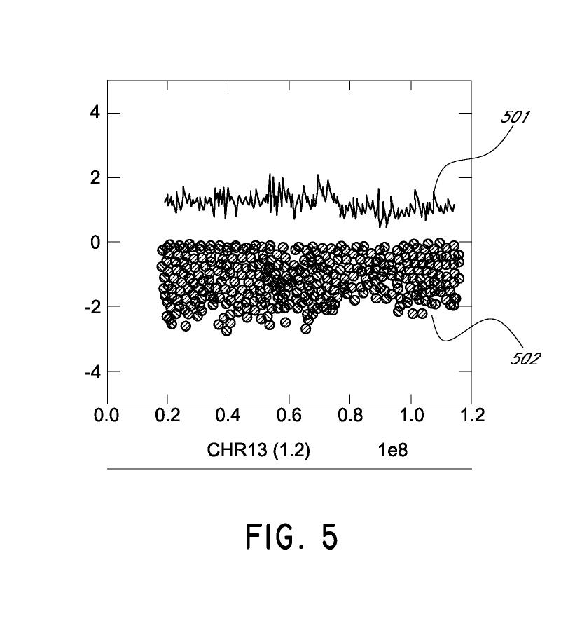

FIG. 5 shows the implementation of an embodiment of the present disclosure. The copy number of a sequence of interest, chromosome 13, for example is determined through the methods disclosed herein. The ratio of ch13 copy number to a region elsewhere in the genome is depicted as 501. The relative number of 20 mer oligonucleotide needles hitting the genomic sample analyzed is depicted in 502. This analysis is performed more rapidly and more quantitatively than present methods.

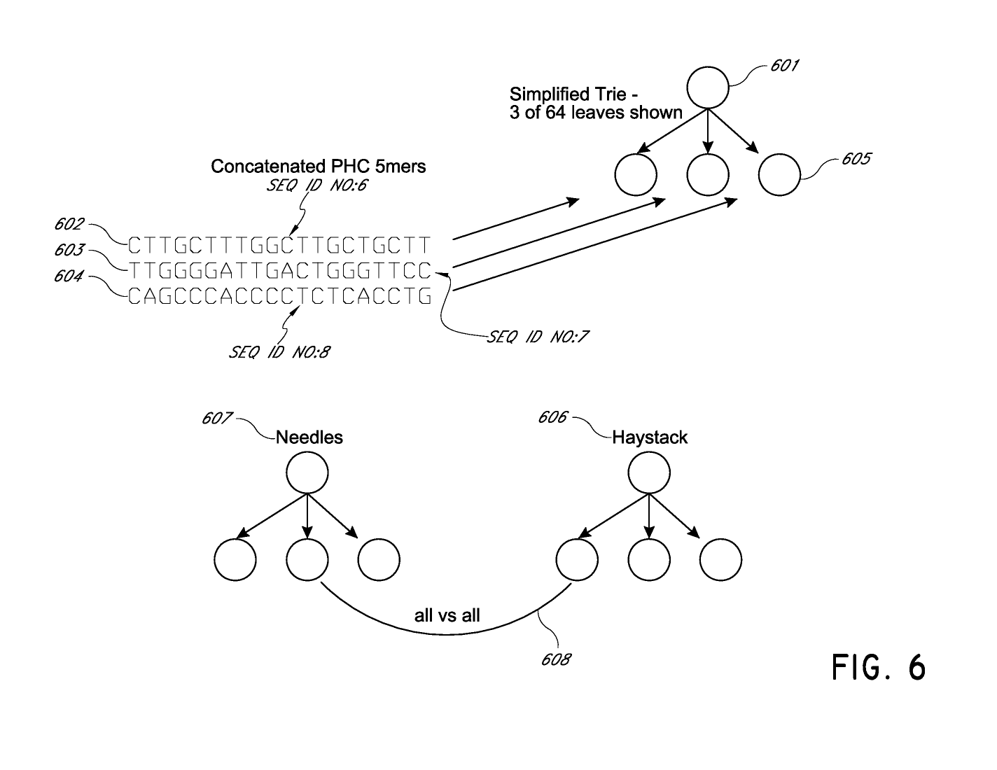

FIG. 6 shows a stylized cartoon of an indexed 20 mer data set. Endpoints corresponding to 20 mer oligonucleotides 602, CTTGCTTTGGCTTGCTGCTT (SEQ ID NO:6); 603 TTGGGGATTGACTGGGTTCC (SEQ ID NO:7); and 604, CAGCCCACCCCTCTCACCTG (SEQ ID NO:8) are shown. The actual trie, it may be noted, has over 16 million leaves at its lowest level, 605. FIG. 6 also shows how in some embodiments of the present disclosure, trie indices of large nucleic acid datasets 606 such as genomic sequence data may be compared with large 20 mer needle data sets 607 in an all versus all comparison 608. This comparison is performed in some embodiments at over times much reduced compared to current methods.

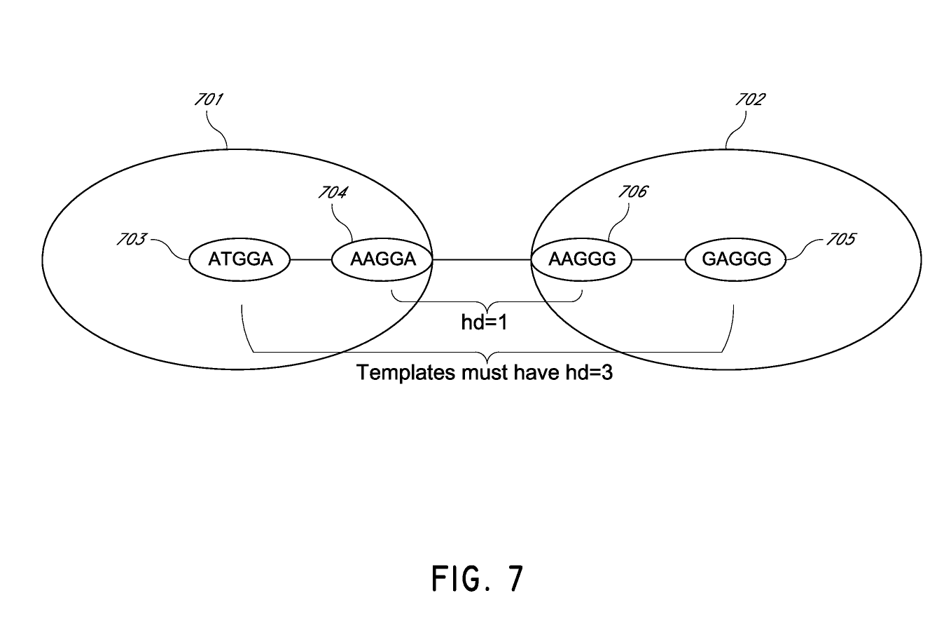

FIG. 7 shows Hamming code distances between members of stylized index classes 701 and 702. PHC 5 mer 703 (‘ATGGA’) is the core of equivalence class 701, which has 15 other 5 mer members differing from 703 by a Hamming code distance of 1. Only one of these members, 704, is shown. Also shown is PHC 5 mer 705 (‘GAGGG’), the core of equivalence class 702. 702 has 15 other 5 mer members differing from 705 by a Hamming code distance of 1. Only one of these members, 706, is shown. 703 and 705 have a Hamming code distance of 3, while 704 and 706 have a Hamming Code distance of 1. This figure illustrates that for non-PHC members of an equivalence class, some members of other equivalence classes may differ by a Hamming Code distance of 1. This necessitates the need to search the 30/64 equivalence classes that may differ include members differing by one from a sequence in an equivalence class.

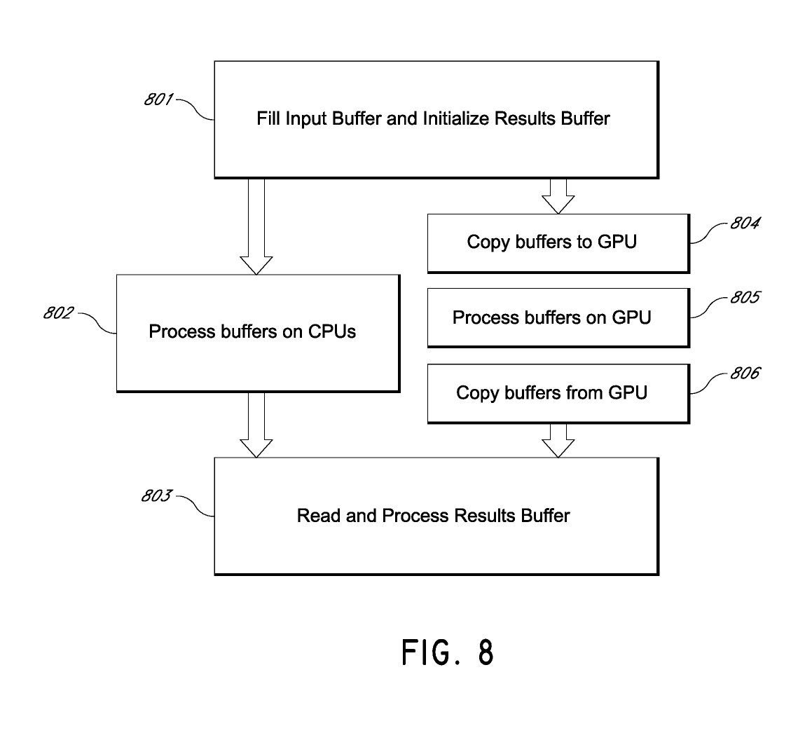

FIG. 8 shows one embodiment of a process for filling input buffers and processing results in a sequence comparison system. The process begins by filling an input buffer and initializing a results buffer at a state 801. In one embodiment the buffers are processed on CPUs at state 802, and are read and their results processed at a state 803. In an alternate embodiment, the buffers are copied to a GPU at a state 804, processed on GPU at a state 805 and copied from a GPU at a state 806, after which they are read and their results processed at the state 803.

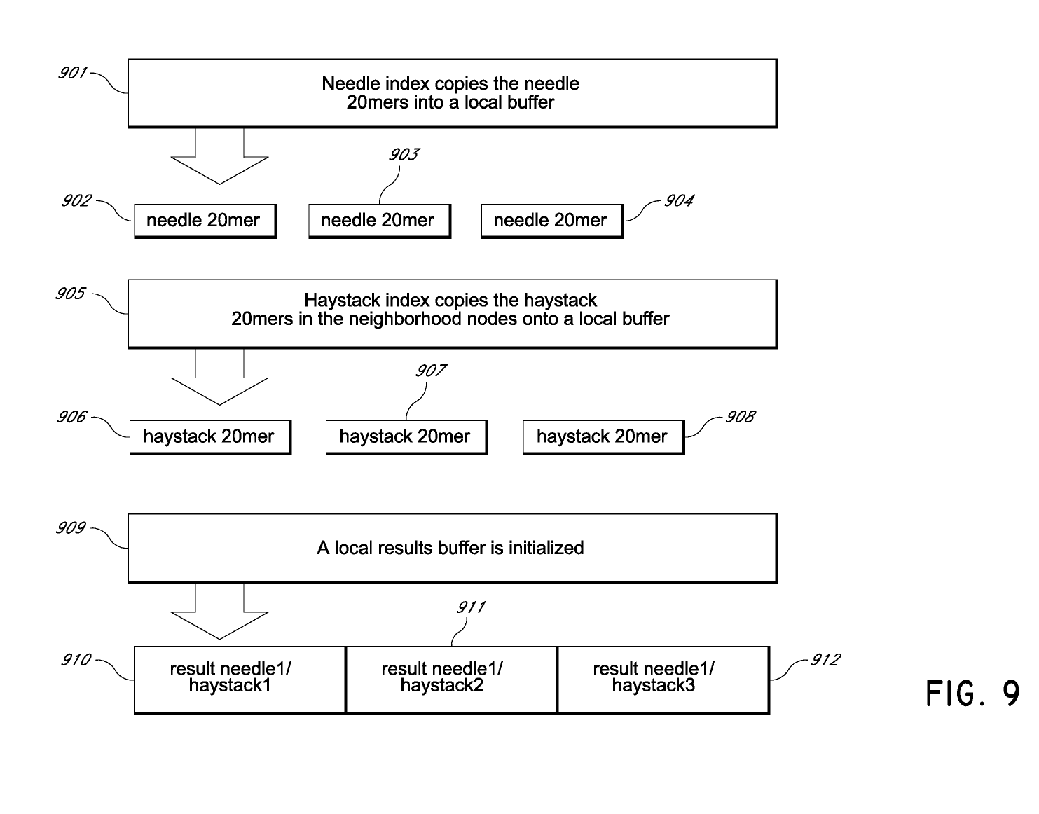

FIG. 9 shows a block diagram of an embodiment of a process for determining results of a search for polynucleotide “needles” within a target “haystack” database of sequences. A needle index copies the needle 20 mers into a local buffer at a state 901, storing needle 20 mers at states 902-904. Similarly, a haystack index copies the haystack 20 mers into a local buffer at a state 905, and storing needle 20 mers at states 906-908. A local results buffer is initialized at a state 909, onto which result comparisons of, for example, needle 1 to haystack 1 (state 910), needle 1 to haystack 2 (state 911) and needle 1 to haystack 3 (state 912) are entered.

### EXAMPLES

### Example 1

### Perfect Hamming Code Template Sequences and Equivalence Class Members

Perfect Hamming code sequences gtaag, cgaac, and tgata are given in Table 1. Each perfect Hamming code 5 mer defines an equivalence class that includes 15 additional 5 mers that differ from the PHC 5 mer by one and only one nucleotide. The single base variation with the PHC 5 mer is highlighted for illustrative purposes.

TABLE 1

| | Equivalence class of |
|---|---|
| PHC | 5-mers covered by the |
| template | template (base differing |
| sequence | from the PHC 5 mer is bold) |
| gtaag | gtaag |
| | ataag ttaag ctaag |
| | gaaag ggaag gcaag |
| | gttag gtgag gtcag |
| | gtatg gtagg gtacg |
| | gtaaa gtaat gtaac |
| cgaac | cgaac |
| | agaac tgaac ggaac |
| | caaac ccaac ctaac |
| | cgtac cggac cgcac |
| | cgatc cgagc cgacc |
| | cgaaa cgaat cgaag |
| tgata | tgata |
| | agata ggata cgata |
| | taata ttata tcata |
| | tgtta tggta tgcta |
| | tgaaa tgaga tgaca |
| | tgatt tgatg tgatc |

### Example 2

### Concatenated PHC 5Mers and Equivalence Class Members

The PHC 5 mers gtaag, cgaac and tgata were concatenated to from the 15 mer oligonucleotide gtaagcgaactgata. Table 2 shows three example 15 mers which are included in the equivalence class of this concatenated PHC 15 mer.

TABLE 2

| | | | |
|---|---|---|---|
| 15 mer of PHC | gtaag | cgaac | tgata |
| 5 mers | | | |
| (SEQ ID NO: 2) | | | |
| 15 mers in | gtaac | cgacc | tgatc |
| equivalence class | | | |
| (read left to right) | ataag | tgaac | tgaga |
| SEQ ID NOs: 3, 4, | | | |
| and 5) | ggaag | cgagc | agata |

1.-43. (canceled)

44. A computer-based method of searching for a first 20 mer oligonucleotide sequence in a nucleotide data set, wherein said sequence is homologous to and differs by at most one nucleotide from a second 20 mer oligonucleotide target sequence, comprising:
selecting a second 20 mer oligonucleotide target sequence having a sequence which is unique in a reference genomic nucleotide data set and which spans one single nucleotide polymorphism;
determining if the selected second 20 mer sequence is present in a sequence determined from a sequencing reaction initiated on a nucleic acid sample to determine if a 20 mer sequence spanning said one single nucleotide polymorphism is present in the sample;
if said 20 mer sequence is present, determining whether said sequence of said sequencing reaction is identical to said first 20 mer oligonucleotide sequence at said single nucleotide polymorphism; and
generating a report indicating whether said second 20 mer is present in any sequence determined from the sequencing reaction.

45. The method of claim 44, further comprising making a decision to (a) perform further analyses on said nucleic acid sequence if said first 20 mer in said sequenced sample is identical to said second 20 mer or (b) halt further analyses on said nucleic acid sequence if said first 20 mer in said sequenced sample is not identical to said second 20 mer.

46. A method of aligning a first 20 mer oligonucleotide sequence to all 20 mer fragments of a nucleotide data set comprising:
indexing said dataset into perfect Hamming code 5 mers, wherein each said perfect Hamming code 5 mer has a set of 15 5 mers which form an equivalence class such that each of said 15 additional members of said class differs from said perfect Hamming code 5 mer by a Hamming code distance of 1, and wherein each said perfect Hamming code 5 mer differs from all other perfect Hamming code 5 mers by at least a Hamming code distance of 3;
configuring said indexed 5 mers into a trie having a branching factor of 64 such that each leaf of said trie represents the perfect Hamming code 5 mers of said dataset and the equivalence class it defines;
concatenating said trie such that each leaf of said index is a trunk of a second level indexed 5 mer trie, each leaf of said second level trie is a trunk of a third level trie and each leaf of said third level trie is a trunk of a fourth level trie, such that said concatenated trie has a depth of 4 and 64⁴ leaves at its fourth level, and such that each 20 mer of said nucleic acids sample maps to only one path from a 4^{th} level leaf to the first level trunk of said trie, wherein each unique trie path corresponds to a concatenation of four consecutive perfect Hamming code 5 mers to form a 20 mer fragment of said trie path;
assigning all 20 mer fragments of said nucleotide data set to a unique trie path;
identifying said trie path corresponding to said 20 mer oligonucleotide sequence; and
comparing said 20 mer fragments of said trie path to said 20 mer oligonucleotide sequence, wherein said comparing comprises:
comparing positions 1-5 of said 20 mer oligonucleotide sequence to the thirty equivalence classes at level 1 having at least on member which differs from at least one member of the equivalence class of the 5 mer oligonucleotide by a Hamming distance of 1,
comparing positions 6-10 of said 20 mer oligonucleotide sequence to the thirty equivalence classes at level 2 having at least on member which differs from at least one member of the equivalence class of the 5 mer oligonucleotide by a Hamming distance of 1,
comparing positions 11-15 of said 20 mer oligonucleotide sequence to the thirty equivalence classes at level 3 having at least on member which differs from at least one member of the equivalence class of the 5 mer oligonucleotide by a Hamming distance of 1,
comparing positions 16-20 of said 20 mer oligonucleotide sequence to the thirty equivalence classes at level 4 having at least on member which differs from at least one member of the equivalence class of the 5 mer oligonucleotide by a Hamming distance of 1, and
comparing said 20 mer oligonucleotide to the concatenated oligonucleotide comprising the four perfect Hamming code 5 mers,
such that all possible oligonucleotide sequences having a Hamming code distance of at most 1 from a 20 mer oligonucleotide sequence due to having one single nucleotide polymorphism relative to the 20 mer oligonucleotide are analyzed through the selection and comparison of 121 out of a total of over 16 million equivalence classes in a four level index of concatenated 5 mers.

47. The method of claim 46, wherein said first 20 mer oligonucleotide maps to a unique region of a genomic reference sequence and wherein said oligonucleotide spans a single nucleotide polymorphic site.

48. The method of claim 47, further comprising populating said equivalence classes with sequence information from a nucleic acid sample sequence.

49. The method of claim 48, further comprising discarding said sample if said 20 mer oligonucleotide sequence is not identically present in said sample sequence.

50. The method of claim 48, wherein said nucleic acid sample sequence comprises sequence from an incompletely sequenced nucleotide sample.

51. The method of claim 50, wherein said nucleotide sample is being sequenced concurrently with execution of said method.

52. The method of claim 47, wherein said method further determines whether said nucleic acid data set and said 20 mer oligonucleotide sequence are derived from different sources.

53. The method of claim 47, wherein said 20 mer oligonucleotide sequence is determined by hybridization of a nucleic acid sample to a nucleic acid probe complementary to said 20 mer oligonucleotide at said single nucleotide polymorphic site.

54. The method of claim 53, wherein said 20 mer oligonucleotide sequence is determined using a microarray hybridization.

55. The method of claim 47, wherein said 20 mer oligonucleotide sequence is determined using a sequencing method.

56. The method of claim 47, wherein a region comprising said oligonucleotide single nucleotide polymorphic site is amplified in a polymerase chain reaction.

57. The method of claim 46, wherein said 20 mer oligonucleotide maps to a region that undergoes copy number variation in a population, and wherein hits to sequence identical to said 20 mer oligonucleotide are indicative of a copy number of said region in said sample.

58. The method of claim 57, further comprising searching said nucleic acid sample using a second 20 mer oligonucleotide sequence that is homologous to a region having a copy number that does not co-vary with said region of claim 15 that undergoes copy number variation.

59. An oligonucleotide data set comprising oligonucleotide sequences, wherein said oligonucleotide sequences are unique in a reference nucleotide data set; each of said oligonucleotide sequences spanning a single nucleotide polymorphic variant within said reference nucleotide data set, said oligonucleotides are separated from one another in sequence by a Hamming Code distance of at least 3, and wherein each of said oligonucleotides has a length of five nucleotides, an integer multiple of five nucleotides, 21 nucleotides or an integer multiple of 21 nucleotides.

60. The oligonucleotide data set of claim 59, wherein said oligonucleotides each have a length of 20 nucleotides.

61. A computer based system for aligning a 20 mer oligonucleotide sequence to all 20 mer fragments of a nucleotide dataset comprising:
an index component comprising perfect Hamming code 5 mers, wherein each said 5 mer has a set of 15 5 mers which form an equivalence class such that each additional member of said class differs from said perfect Hamming code 5 mer by a Hamming code distance of 1, and wherein each said perfect Hamming code 5 mer differs from all other perfect Hamming code 5 mers by at least a Hamming code distance of 3;
an iterative trie component of said perfect Hamming code 5 mers having a branching factor of 64 such that each leaf of said trie represents a perfect Hamming code 5 mer of said dataset and the equivalence class it defines, and such that each leaf of a first level of said trie is the trunk of a second indexed 5 mer trie, each leaf of said second level trie is the trunk of a third level trie trunk, and each leaf of said third level trie is the trunk of a fourth level trie, such that said trie has a depth of 4 levels and 64⁴ leaves, and such that each 20 mer of said nucleic acids sample maps to only one path from a 4^{th} level leaf to the first level trunk of said trie;
an assignment component that assigns all 20 mer fragments of said nucleotide data set to a unique trie path corresponding to said 20 mer fragments;
an identification component identifying said trie path corresponding to said 20 mer oligonucleotide sequence;
a comparing component for:
comparing said 20 mer fragments of said trie path to said 20 mer oligonucleotide sequence,
comparing a first five positions of said 20 mer oligonucleotide sequence to the thirty equivalence classes at level 1 having at least on member which differs from at least one member of the equivalence class of the 20 mer oligonucleotide by a Hamming distance of 1,
comparing a second five positions of said 20 mer oligonucleotide sequence to the thirty equivalence classes at level 2 having at least on member which differs from at least one member of the equivalence class of the 20 mer oligonucleotide by a Hamming distance of 1,
comparing a third five positions of said 20 mer oligonucleotide sequence to the thirty equivalence classes at level 3 having at least on member which differs from at least one member of the equivalence class of the 20 mer oligonucleotide by a Hamming distance of 1, and
comparing a fourth five positions of said 20 mer oligonucleotide sequence to the thirty equivalence classes at level 4 having at least on member which differs from at least one member of the equivalence class of the 20 mer oligonucleotide by a Hamming distance of 1,
such that all possible oligonucleotide sequences having a Hamming code distance of at most 1 from a 20 mer oligonucleotide sequence are analyzed through the selection of 121 out of a total of over 16 million equivalence classes in a four level index of concatenated 5 mers; and
a report module capable of reporting the results of said evaluation to a user.

62. The system of claim 61, comprising a control module capable of controlling a nucleic acid generating sequence apparatus in contact with said sample.

63. The system of claim 61, wherein said system has a component to receive data from a sequencing reaction.
